(19) United States
(12) Patent Application Publication
(10) Pub. No.: US 2021/0013929 A1
(43) Pub. Date: Jan. 14, 2021

(54) ELECTRICAL NODE WITH MONITORING FEATURE

(71) Applicant: 3M INNOVATIVE PROPERTIES COMPANY, Saint Paul, MN (US)

(72) Inventor: William Bedingham, Woodbury, MN (US)

(73) Assignee: 3M INNOVATIVE PROPERTIES COMPANY, Saint Paul, MN (US)

(21) Appl. No.: 16/924,773

(22) Filed: Jul. 9, 2020

Related U.S. Application Data
(60) Provisional application No. 62/872,345, filed on Jul. 10, 2019.

(51) Int. Cl. H04B 3/54; A61B 5/0205; A61B 5/00; H02M 7/537

(52) U.S. Cl. CPC H04B 3/548; A61B 5/0205; H04B 2203/5483; H02M 7/537; H04B 2203/547; A61B 5/002

(57) ABSTRACT

A first electrical node configured to monitor a digital signal is disclosed. The first electrical node includes a first connector and a second connector. The first electrical node includes a processor configured to receive a digital signal from the second connector. The processor is configured to determine whether a portion of the digital signal is intended for the first electrical node. The processor is configured to convert the portion of the digital signal to an analog signal based on whether the portion is intended for the first electrical node without affecting the digital signal received.

### TECHNICAL FIELD

The present disclosure relates generally to electrical nodes with a functionality to monitor sensor signals.

### BACKGROUND

Sensor devices are used in medical setting for measuring a patient's vital signs (e.g. electrocardiogram (ECG), oxygen saturation (SPO2), temperature, blood pressure, etc.). For example, a pulse oximeter provides an indirect measurement of the oxygen saturation of a patient's blood. An ECG electrode monitors the electrical activity of the heart over time. These sensor devices are generally attached to the patient body. Sensor data is provided to a medical monitor.

Medical monitors are used to monitor vital signs in multiple locations (e.g. intensive care unit (ICU), operating room, radiology) and during transport (e.g. transport within hospital, transport in ambulance, transport in helicopter). Traditional medical monitors have cables attached to each sensor. Each sensor cable is plugged into the appropriate input port of the medical monitor. Wireless sensing of vital signs offers the advantage of sending the patient's sensor data wirelessly to the monitor. However, conventional medical monitors may not be designed to receive wireless sensor streams. Consequently, a wireless receiver module may be needed to receive the wireless sensor signals and process them into a format that the monitor can correctly process. Multiple cables can also be used to deliver the wireless sensor signals to each port in the conventional medical monitor. Further, additional cables can also deliver power, adding to the number of cables in the monitor.

### SUMMARY

At least some aspects of the present disclosure direct to a first electrical node for forming daisy-chain buses with simultaneous serial data transmission and power transmission. The first electrical node includes a housing, a first connector adjacent the housing, and a second connector adjacent the housing. The first connector is operable to mate with a computing device. The second connector includes a second connector power contact and a second connector data contact. The first electrical node includes a controller circuit. The controller circuit includes a digital-to-analog circuit that is electrically coupled to the first connector, a processor, and a memory communicatively coupled to the processor. The processor is configured to receive a digital signal from the second connector data contact. The processor is configured to determine whether a portion of the digital signal is intended for the first electrical node. The processor is configured to convert, with the digital-to-analog circuit, the portion of the digital signal to an analog signal based on whether the portion is intended for the first electrical node without affecting the digital signal received.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system in accordance with one embodiment.

FIG. 2 illustrates a system in accordance with one embodiment.

FIG. 3 illustrates a first electrical node in accordance with one embodiment.

FIG. 4 illustrates a system in accordance with one embodiment.

FIG. 5 illustrates a system in accordance with one embodiment.

FIG. 6 illustrates a first electrical node in accordance with one embodiment.

FIG. 7 illustrates a method in accordance with one embodiment.

FIGS. 8A and 8B illustrate a system in accordance with one or more embodiments.

### DETAILED DESCRIPTION

“mammalian subject” refers to any animal of the Mammalia, a large class of warm-blooded vertebrates having mammary glands in the female, a thoracic diaphragm, and a four-chambered heart. The class includes whales, carnivores, rodents, bats, primates, humans, etc.

“physiological parameters” refers to any measurement relating to a bodily function of a mammal. Examples include temperature, heart rate, ECG, blood pressure, blood flow, blood volume, respiration, skin condition, shivering, blood sugar, or combinations thereof.

“negative supply voltage” refers to a voltage negative relative to ground

“positive supply voltage” refers to a voltage positive relative to ground

“cable” refers to bundle of one or more wires

“medical monitor” refers to an electronic medical device that includes one of more monitoring sensors, a processing component(s), and a screen display (also called a “monitor”) that provide and record for medical professionals a patient's medical vital signs (body temperature, blood pressure, pulse rate and respiratory rate) or measurements of the activity of various body organs. Examples include ECG monitors, anesthesia monitors, or EKG monitors.

“power supply” refers to an electrical device that supplies electric power to an electrical load, preferably the output voltage is no greater than 5 volts.

Aspects of the present disclosure relate to an electrical node configured to monitor a digital signal from one connector, determine whether a portion of the digital signal is intended for the electrical node, and convert the portion to an analog signal for transmission to another connector within the electrical node.

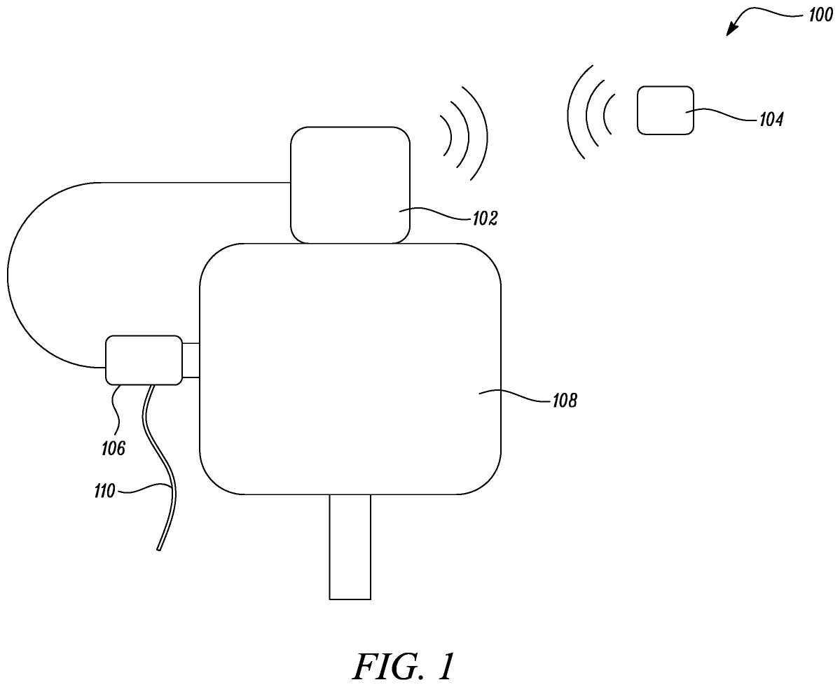

FIG. 1 illustrates a system 100 in accordance with one embodiment. The system 100 includes a receiver device 102. In one embodiment, the receiver device 102 is configured to receive one or more physiological parameters of a mammalian subject wirelessly. Examples of physiological parameters include temperature, heart rate, ECG, blood pressure, blood flow, blood volume, respiration, skin condition, shivering, blood sugar, oxygen saturation, pulse rate, respiration rate, fractional arterial oxygen saturation, total hemoglobin, plethysmograph variability index, methemoglobin, carboxyhemoglobin, perfusion index, oxygen content, or combinations thereof.

In one embodiment, the receiver device 102 is communicatively coupled to a sensor device 104. The receiver device 102 may communicate with the sensor device 104 wirelessly. The receiver device 102 may include electronics to provide one or more of short-range wireless communication interfaces, such as interfaces conforming to a known communications standard, for example, a Bluetooth standard, Bluetooth Low Energy (BLE), near field communication protocol, Institute of Electrical and Electronics Engineers (IEEE) 802 standards (e.g., IEEE 802.11), a ZigBee or similar specification, such as those based on the IEEE 802.15.4 standard, or other public or proprietary wireless protocol. The sensor device 104 may be configured to monitor the one or more physiological parameters and provide the physiological parameters to the receiver device 102. In one embodiment, the system 100 includes a plurality of sensor devices 104. The sensor devices 104 may include, for example, electrocardiography (ECG) electrodes, oximeter sensors, body temperature sensors, blood pressure sensors, acoustic sensors, humidity sensors, light sensors, or combinations thereof.

The sensor device 104 may send data wirelessly from a single physiological sensor, or from multiple physiological sensors. Additionally, there can be multiple sensor devices 104 communicating to the same receiver device 102.

The system 100 includes a first electrical node 106 that is communicatively coupled to the receiver device 102. Referring to FIG. 1, the first electrical node 106 is connected to the receiver device 102 via a wired connection. The first electrical node 106 is configured to receive a digital signal from the receiver device 102. The digital signal may be associated with the data provided by the sensor device 104. The first electrical node 106 may process at least a portion of the received digital signal and provide the output to a computing device 108. In one embodiment, the computing device 108 is a medical monitor, particularly a medical monitor configured to receive only data transmitted via cables from a sensor and having no on-board wireless communication devices. In other embodiments, the computing device 108 may be any device known in the art that receives multiple inputs corresponding to multiple input ports on the computing device 108.

The first electrical node 106 is provided with processing circuitry to process at least a portion of the digital signal and convert the portion of the digital signal to an analog signal to be fed into the computing device 108. The analog signal may be an ECG signal, a SPO2 signal, a temperature signal, a blood pressure signal, and combinations thereof. In some embodiments, the first electrical node 106 has one or more connectors to electrically couple with one or more input ports on the computing device 108. The computing device 108 may be configured to store and further process the received data, to display information indicative of or derived from the received data, and to transmit information including displays, alarms, alerts, and notifications to patient monitoring systems and/or multi-patient monitoring systems. The computing device 108 may be a bedside monitor located on a table or a support at the side of the patient's bed or a transport monitor to monitor physiological parameters when transporting patients.

Referring to FIG. 1, the system 100 includes a first cable 110 to electrically couple the first electrical node 106 to a power supply. The power supply may be configured to provide low power using, for example, a direct current (DC) power source generating voltage of 1 to 5 volts or a micro-universal serial bus (USB) connection.

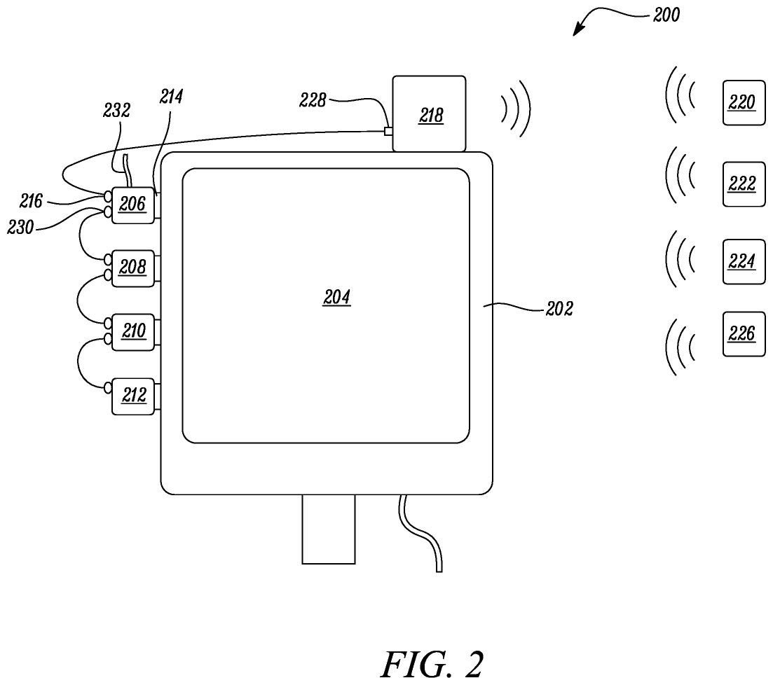

FIG. 2 illustrates a system 200 in accordance with one embodiment. The system 200 includes a computing device 202, for example, a medical monitor. The computing device 202 may be configured to process the received data to display information indicative of or derived from the received data. The computing device 202 includes a display 204 that displays one or more physiological parameters of the mammalian subject. Referring to FIG. 2, the system 200 includes a first electrical node 206, a second electrical node 208, a third electrical node 210, and a fourth electrical node 212 that are electrically coupled to respective input ports of the computing device 202 to provide the received data.

The first electrical node 206 has a first connector 214 operable to mate with the computing device 202. The first electrical node 206 has a second connector 216. The second connector 216 may be electrically coupled to a receiver device 218. The second connector 216 includes a second connector power contact and a second connector data contact. For example, the second connector 216 may be a USB connector (e.g., microUSB, USB type A or USB type C). In one embodiment, the second connector power contact is configured to receive power through a second cable 232 electrically coupled with a power supply. The second connector data contact may receive a digital signal from the receiver device 218. In one embodiment, the second connector data contact receives a digital signal when the second connector power contact receives power from the power supply. In another embodiment, the second connector data contact does not receive a digital signal when the second connector power contact does not receive power from the power supply. In one embodiment, the power and data can be independent and/or mutually exclusive. For example, the second connector data contact may not receive a digital signal when the second connector power contact receives power from the power supply.

The first electrical node 206 is configured to receive digital signal from the receiver device 218. The receiver device 218 may be configured to wirelessly receive sensor data from sensor devices 220, 222, 224, 226. In one embodiment, the sensor data received from the sensor devices 220, 222, 224, 226 is processed by the first, second, third and/or fourth electrical nodes 206, 208, 210, 212.

In an example embodiment, the sensor device 220 is an ECG electrode that wirelessly provides ECG data to the receiver device 218. The receiver device 218 provides a digital signal including the ECG data to the first electrical node 206. The first electrical node 206 can monitor the digital signal and determine whether a portion of the digital signal is intended for the first electrical node 206. Upon affirmative determination, the first electrical node 206 processes the ECG data from the digital signal and provides the ECG data to the first connector 214 (e.g., as an analog signal) and the digital signal is subsequently passed on to the second electrical node 208.

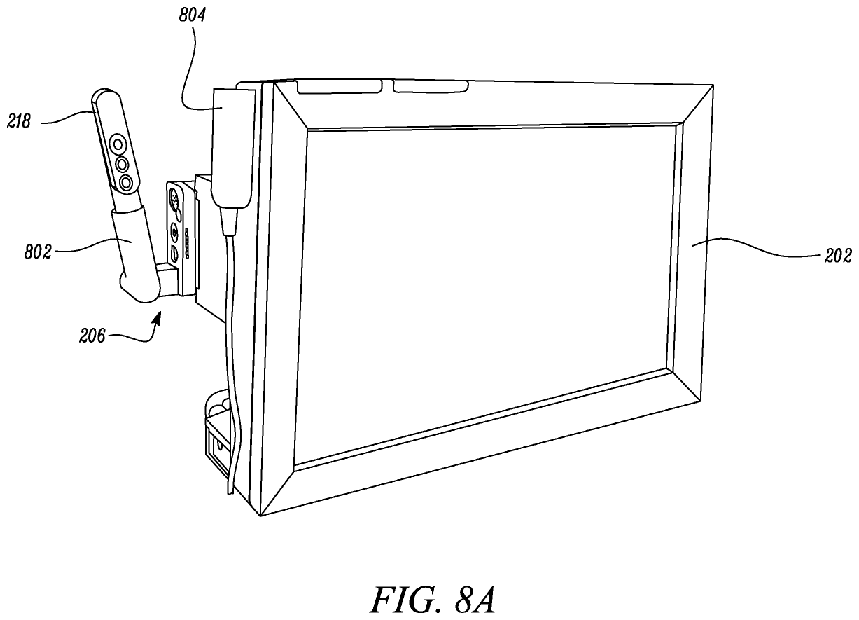

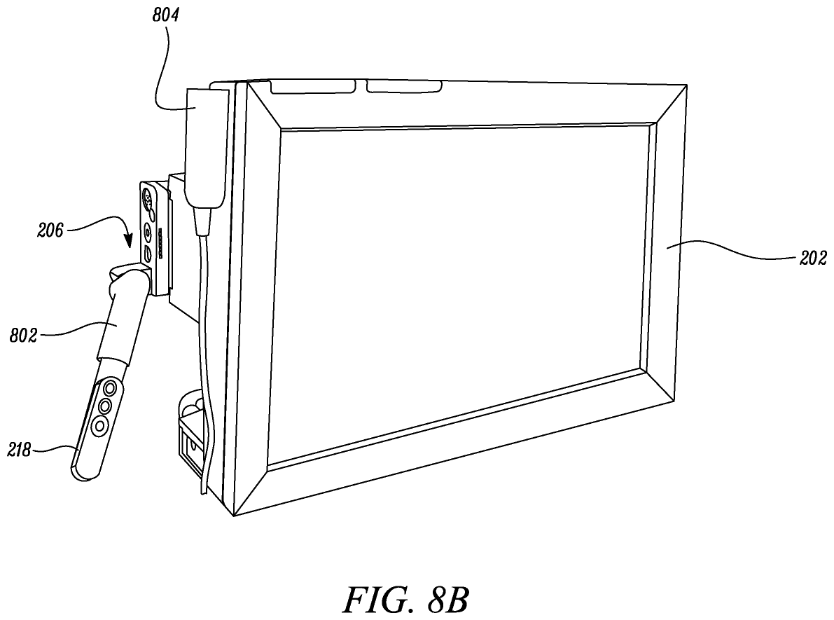

Referring to FIG. 2, the receiver device 218 has a receiver connector 228 that couples to the first electrical node 206. In one embodiment, the receiver device 218 is powered through the first electrical node 206 via the receiver connector 228. The receiver device 218 may have a receiver housing and wireless communication circuitry. The receiver device 218 may have digital to analog conversion capability. The receiver connector 228 may be configured to be removable from the second connector 216. The receiver housing may be arranged to nestle inside of the housing of the first electrical node 206 such that the receiver connector 228 mates with the second connector 216. In one embodiment, the receiver device 218, when nestled inside of the housing, does not cause mechanical interference with any other nodes in a linear configuration. Further, as shown in FIGS. 8A and 8B, the receiver device 218 may be configured to be oriented in a vertical orientation when the first electrical node 206 is inserted into the computing device 202. In one embodiment, the first electrical node 206 includes a first cradle 802 that is physically dimensioned to receive at least a part of the receiver device 218. The first cradle 802 can be configured to extend away from the computing device 202 and the receiver device 218 is attached to the first cradle 802 such that the receiver device 218 remains attached to the first cradle 802 in an upright position. The first electrical node 206 can have a second connector 216 that is configured to form an electrical connection with and releasably detach from the receiver device 218. In one embodiment, a second cradle 804 can be coupled to a power source to power the receiver device 218. The second cradle 804 may be physically dimensioned to receive at least a part of the receiver device 218. The receiver device 218 can be wirelessly linked to the first electrical node 206.

The first electrical node 206 may receive digital signal from the second connector data contact. The first electrical node 206 is configured to process the digital signal and provide the processed signal to the computing device 202. For example, the first electrical node 206 may process the digital signal having ECG data and provide the processed signal to the first connector 214 of the computing device 202. The first electrical node 206 is provided with processing circuitry to determine whether a portion of the digital signal received from the receiver device 218 is intended for the first electrical node 206. The first electrical node 206 may extract packet header, packet footer, data type, and/or other characteristics of the digital signal and process the extracted information to determine whether the portion of the signal is intended for the first electrical node 206. Upon an affirmative determination, the first electrical node 206 converts the portion of the digital signal to an analog signal and provides the analog signal to the computing device 202 via the first connector 214.

In one embodiment, the first electrical node 206 broadcasts the entire digital signal to the second electrical node 208 for further processing. The second electrical node 208 may extract information from packet headers of the digital signal and broadcast the entire digital signal to the third electrical node 210. Referring to FIG. 2, the first electrical node 206 includes a third connector 230 to connect the first electrical node 206 with the second electrical node 208. The third connector 230 may include a third connector power contact and a third connector data contact. The second connector power contact may be electrically coupled to the third connector power contact. In one embodiment, the second connector power contact or the third connector power contact is a positive supply voltage, negative supply voltage, ground wire, or combinations thereof. In some embodiments, the second connector data contact or the third connector data contact includes a read wire, a write wire, a common wire or combinations thereof.

The second electrical node 208 may have components similar to those of the first electrical node 206. The second electrical node 208 may process another portion of the digital signal corresponding to the input port of the computing device 202 with which the second electrical node 208 is connected. For example, the second electrical node 208 may be connected to an oxygen saturation (SPO2) port of the computing device 202 and hence, the second electrical node 208 may convert the portion of the digital signal related to oxygen saturation into an analog signal. The analog signal may be provided to the computing device 202. In one embodiment, the first connector data contact of the second electrical node 208 is configured to electrically couple to the second connector data contact of the first electrical node 206 such that digital signals from the receiver device 218 at the first electrical node 206 are received by the second electrical node 208. The system 200 may include a second cable to electrically couple the third connector 230 of the first electrical node 206 with a second connector of the second electrical node 208. The second electrical node 208 may provide the entire digital signal to the third electrical node 210 for further processing.

In one embodiment, the sensor 222 can be an SPO2 sensor. SPO2 data from sensor 222 can be received by receiver device 218 along with ECG data from sensor device 220. In one embodiment, data from sensor 222 and sensor device 220 can be commingled and carried over the same wire. For example, ECG data and SPO2 data can be packetized and arranged such that ECG data and SPO2 data are interspersed. The first electrical node 206 can read the ECG data from the digital signal and provide the ECG data to the first connector 214 (which corresponds to a port on computing device 202). The second electrical node 208 can read the SPO2 data from the digital signal and provide the SPO2 data from the digital signal to the computing device 202 at an input corresponding to the SPO2 connection.

The third electrical node 210 may have components similar to those of the first electrical node 206. Further, the third electrical node 210 may process yet another portion of the digital signal corresponding to the input port of the computing device 202 with which the third electrical node 210 is connected. For example, the third electrical node 210 may be connected to temperature port of the computing device 202 and hence, the third electrical node 210 may convert the portion of the digital signal related to temperature into an analog signal. The analog signal may be provided to the computing device 202. In one embodiment, the third connector data contact of the third electrical node 210 is configured to electrically couple to the second connector data contact of the first electrical node 206 such that digital signals from the receiver device 218 at the first electrical node 206 are received by the third electrical node 210. The system 200 may include a third cable to electrically couple a third connector of the second electrical node 208 with a second connector of the third electrical node 210. The second electrical node 208 may provide the entire digital signal to the fourth electrical node 212 for further processing.

The fourth electrical node 212 may have components similar to those of the first electrical node 206. Further, the fourth electrical node 212 may process yet another portion of the digital signal corresponding to the input port of the computing device 202 with which the fourth electrical node 212 is connected. For example, the fourth electrical node 212 may be connected to a blood pressure port of the computing device 202 and hence, the fourth electrical node 212 may process the portion of the digital signal related to blood pressure. As shown in FIG. 2, the first electrical node 206 enables formation of daisy-chain buses with the second, third, and fourth electrical nodes 208, 210, 212 for simultaneous serial data transmission and power transmission.

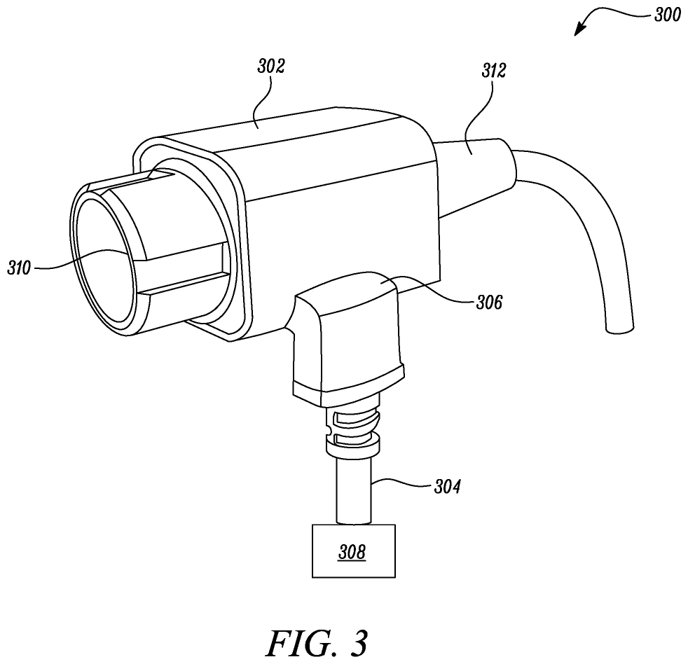

FIG. 3 illustrates a first electrical node 300 in accordance with one embodiment. The first electrical node 300 includes a housing 302. The first electrical node 300 includes a first cable 304 to electrically couple a second connector 306 to a power supply 308. In one embodiment, digital signals may not be transmitted through the first cable 304. The first electrical node 300 includes a first connector 310 operable to mate with an input port on a computing device. The first connector 310 may be a male connector or a plug (as shown in FIG. 3) operable to mate with a corresponding female connector on the computing device. The first electrical node 300 includes a fourth connector 312 within the housing 302. The fourth connector 312 is configured to electrically couple to the receiver device 218 (as shown in FIG. 2). In one embodiment, the receiver device 218 provides the digital signal to the first electrical node 300 via the fourth connector 312.

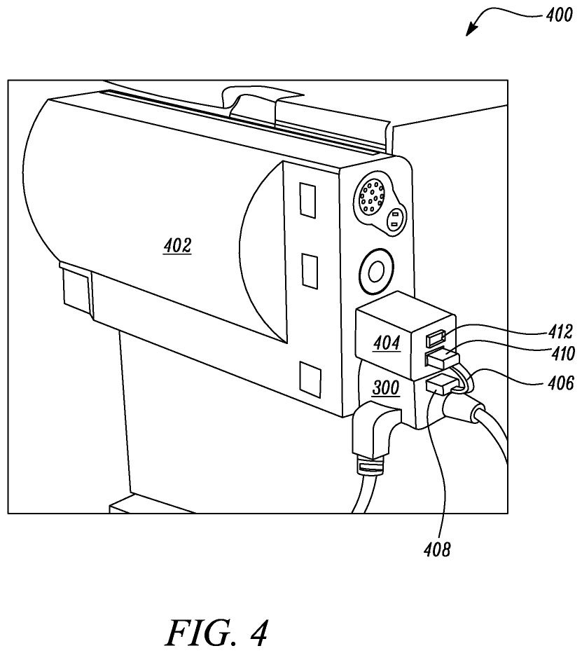

FIG. 4 illustrates a system 400 in accordance with one embodiment. The system 400 includes a first electrical node 300 configured to form daisy-chain buses with simultaneous data transmission and power transmission. The first electrical node 300 processes the received digital signal (from the receiving device) to determine whether a first portion of the digital signal is intended for the first electrical node 300. Upon an affirmative determination, the first electrical node 300 processes the first portion of the digital signal and provide the processed signal to the input port of a computing device 402 (e.g., medical monitor) with which the first electrical node 300 is connected. If the first electrical node 300 determines that the digital signal is not intended for the first electrical node 300, the digital signal is not processed by the first electrical node 300. In one embodiment, the digital signal is received by the first electrical node 300 and then forwarded to the next node for processing. In other embodiments, the digital signal and/or power are on a signal bus and all nodes receive and process the digital signal and/or power at the same time.

The system 400 includes a second electrical node 404 connected to the computing device 402. The second electrical node 404 is connected to the first electrical node 300 via a second cable 406. The second cable 406 allows simultaneous transmission of data and power between the first and second electrical nodes 300, 404. The first electrical node 300 may transmit the digital signal to the second electrical node 404 via a third connector 408. Specifically, the third connector 408 of the first electrical node 300 is electrically coupled with a second connector 410 of the second electrical node 404 via the second cable 406. The second electrical node 404 may process the digital signal received from the first electrical node 300 to determine whether a second portion of the digital signal is intended for the second electrical node 404. Upon an affirmative determination, the second electrical node 404 may process the second portion of the digital signal corresponding to the input port of the computing device 402 with which the second electrical node 404 is connected. Subsequently, the second electrical node 404 may transmit the digital signal to the next node (not shown in FIG. 4) via a third connector 412 of the second electrical node 404. In one embodiment, the third connector 412 of the second electrical node 404 is electrically coupled with a second connector of the next electrical node. The arrangement of the first and second electrical nodes 300, 404 may be used for simultaneous data and power transmission in the daisy chain.

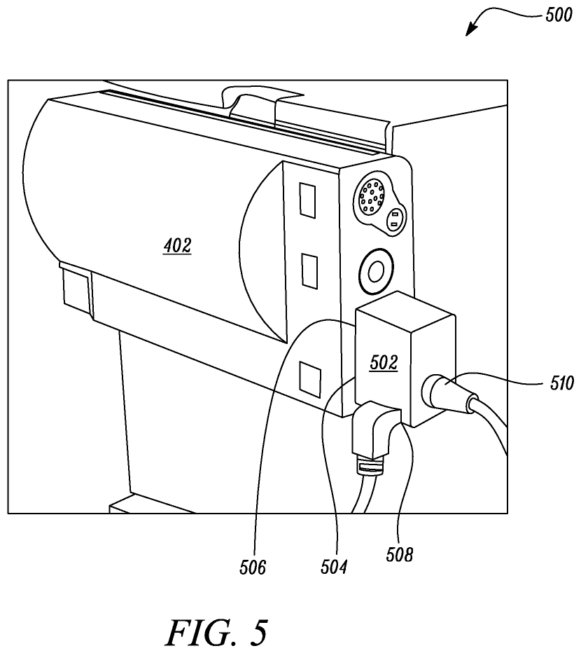

FIG. 5 illustrates a system 500 in accordance with one embodiment. The system 500 includes a first electrical node 502 having a first connector 504 to connect to an input port of the computing device 402. In one embodiment, the first electrical node 502 includes a fifth connector 506 to connect to another input port of the computing device 402. For example, the first connector 504 may be connected to an ECG port and the fifth connector 506 may be connected to the SPO2 port of the computing device 402. An aspect of the first electrical node 502 is that the circuitry for connector 504 and the connector 506 are within the same housing. The first electrical node 502 includes a second connector 508 to receive power from a power supply. The first electrical node 502 may receive the digital signal from the second connector 508 or a fourth connector 510. Specifically, the first electrical node 502 may receive a digital signal from the second connector data contact. The fourth connector 510 may be configured to receive digital signal from the receiver device 218 (shown in FIG. 2). The first electrical node 502 determines whether a portion of the digital signal is intended for at least one of the fifth connector 506 and the first connector 504. Upon an affirmative determination, the first electrical node 502 processes the portion of the digital signal and provides the analog signal to the computing device 402 via the fifth connector 506 and/or the first connector 504.

In one embodiment, each node may transmit data back to the receiver device 218. The data may include identified port, failure indication, node status, monitor model, and monitor manufacturer. Specifically, receiver device 218 may receive information from each node regarding whether the node is active or inactive.

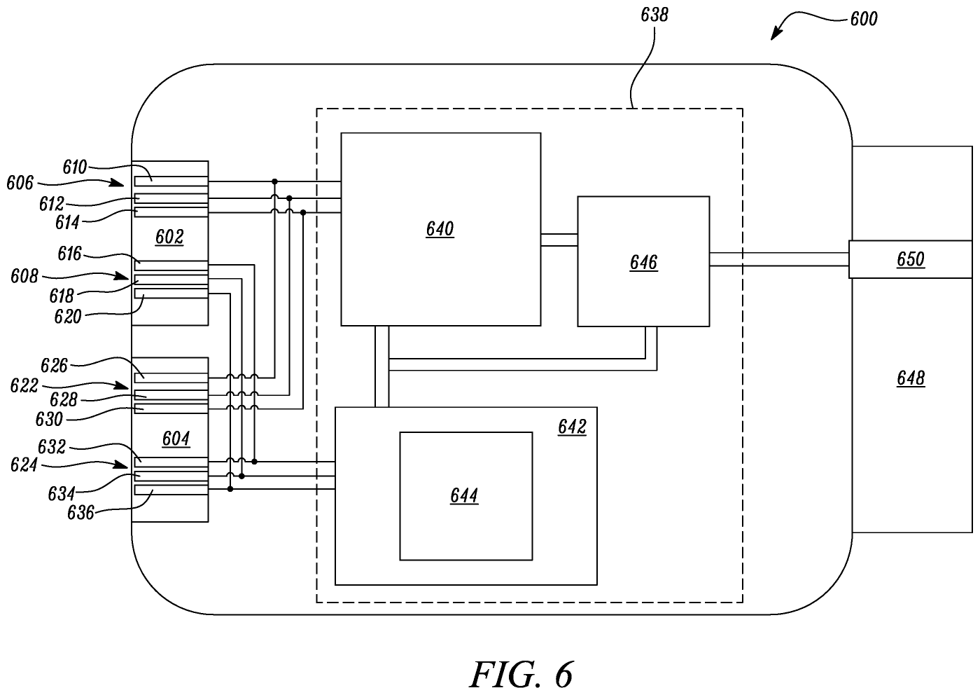

FIG. 6 illustrates a first electrical node 600 in accordance with one embodiment. The first electrical node includes a second connector 602 and a third connector 604. The second connector 602 includes a second connector data contact 606 and a second connector power contact 608. The second connector data contact 606 includes a write wire 610, a read wire 612, and a common wire 614. The second connector power contact 608 includes a positive supply voltage 616, a negative supply voltage 618, and a ground wire 620.

The third connector 604 includes a third connector data contact 622 and a third connector power contact 624. The third connector data contact 622 includes a write wire 626, a read wire 628, and a common wire 630. The third connector power contact 624 includes a positive supply voltage 632, a negative supply voltage 634, and a ground wire 636. The second connector power contact 608 is electrically coupled to the third connector power contact 624. As shown in FIG. 6, the positive supply voltage 616, the negative supply voltage 618, and the ground wire 620 are respectively connected to the positive supply voltage 632, the negative supply voltage 634, and the ground wire 636. In one embodiment, a single wire supports both the write and read functions. The read and write data may be multiplexed on one wire. In another embodiment, a sending node waits for an opportunity to send data on a wire and once the packet transfer is initiated, other nodes wait to send data on that wire until the packet transfer is complete.

The first electrical node 600 includes a controller circuit 638 configured to process the digital signal received at the second connector data contact 606. The controller circuit 638 includes a processor 640 configured to receive the digital signal received from the second connector data contact 606. The processor 640 may include a central processing unit (CPU) to process the received signals. In some cases, the processor 640 may further include a digital signal processor (DSP) to perform signal processing on the received signals, and/or an imaging processor to generate display contents. The processor 640 may include a memory (not shown) that is communicatively coupled to the processor 640. The memory may include instructions that may be executed by the processor 640.

The controller circuit 638 includes a power management circuit 642 electrically coupled to the second connector power contact 608 and the third connector power contact 624. The power management circuit 642 may be configured to draw power from the second connector power contact 608 to power the controller circuit 638. A voltage drop between the second connector power contact 608 and the third connector power contact 624 may be less than 1 volt. In one embodiment, the power management circuit 642 includes a battery 644 as shown in FIG. 6. In other embodiments, the power management circuit 642 includes a capacitor (not shown).

The controller circuit 638 includes a signal converter 646 that is electrically coupled to a first connector 648. The signal converter 646 may be connected to the power management circuit 642 to receive power for its operation. The signal converter 646 may include a digital-to-analog circuit to convert a digital signal into an analog signal. The processor 640 determines whether a portion of the digital signal is intended for the first electrical node 600. The processor 640 may receive the digital signal in the form of a stream of packets. The processor 640 may analyze the packet header, packet footer, data type, or other packet characteristics to determine whether some or all of the received packets are intended for the first electrical node 600. Upon an affirmative determination, the processor 640 may convert the portion of the digital signal to an analog signal using the digital-to-analog circuit of the signal converter 646 without affecting the digital signal received.

In one embodiment, the processor 640 includes other circuitry (such as amplifiers, filters) before the digital-to-analog circuit and/or after the digital-to-analog circuit to provide the appropriate signals at interfaces. For example, for SPO2 measurement, an optical detection circuit provides digital signals from the medical monitor which are converted into analog signals before sending back to the medical monitor.

The analog signal is provided to the medical monitor via a first connector data contact 650 of the first connector 648. In one embodiment, the controller circuit 638 forwards the digital signal received by the first electrical node 300 to the next node through the third connector data contact 622 for processing. In other embodiments, the digital signal is received simultaneously by all nodes (for example, party line transmission) and all nodes extract the information that they need from the digital signal.

In some embodiments, the signal converter 646 includes an analog-to-digital circuit coupled to the first connector data contact 650. The first connector data contact 650 may receive a second analog signal from the medical monitor and provide it to the signal converter 646. The signal converter 646 may convert the second analog signal to a second digital signal using the analog-to-digital circuit. The second digital signal may be transmitted using the second connector data contact 606 and the third connector data contact 622.

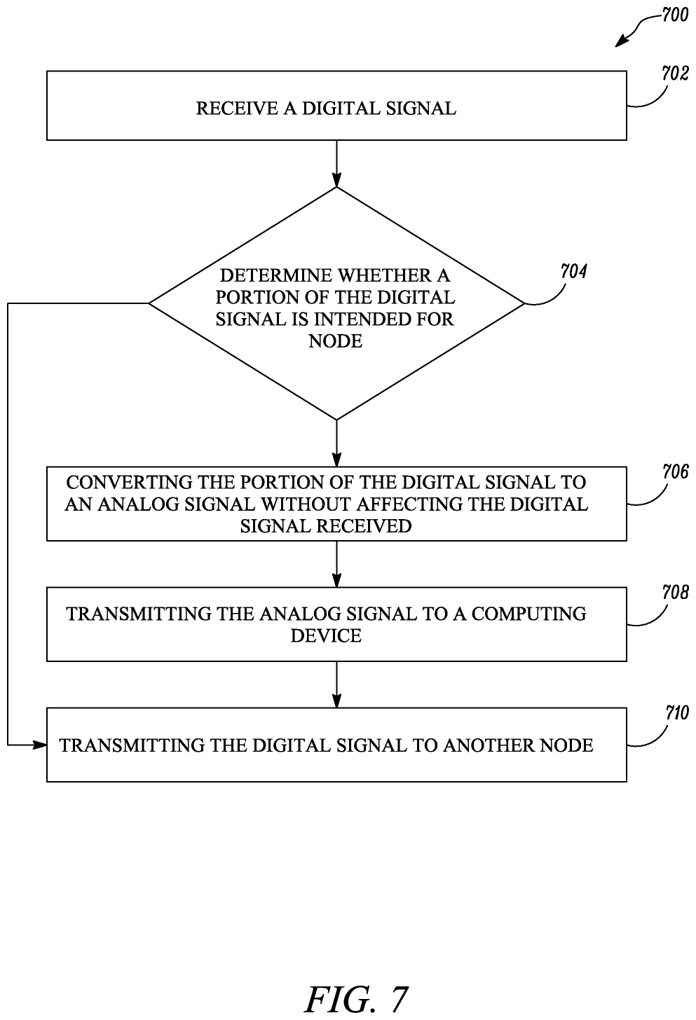

FIG. 7 illustrates a method 700 for monitoring a digital signal in accordance with one embodiment. The method 700 begins at block 702. In block 702, the first electrical node is configured to receive a digital signal via a data contact. The digital signal may be received by the controller circuit or any portion of the controller circuit. In case, multiple nodes are attached to the medical monitor, the digital signal may be forwarded to all the nodes simultaneously on a signal bus.

In block 704, the controller circuit is configured to determine whether a portion of the digital signal is intended for the first electrical node. The controller circuit may analyze the packet header, packet footer, data type, or other packet characteristics to determine whether some or all of the packets of the digital signal are intended for the first electrical node. In at least one embodiment, upon an affirmative determination, the method 700 may continue to block 706. In block 706, the controller circuit may convert the portion of the digital signal to an analog signal without affecting the digital signal received. The digital-to-analog circuit of the signal converter 646 may be used to convert the digital signal to the analog signal.

In block 708, the analog signal is transmitted to the computing device 202. The analog signal may be transmitted via a first connector data contact of the first connector. The computing device may be configured to store and/or further process the received analog signal, to display information indicative of or derived from the received analog signal, and to transmit information including displays, alarms, alerts, and notifications to other systems.

In at least one embodiment, if the determination in block 704 is negative, the method 700 may continue to (optional) block 710. In block 710, the controller circuit may transmit the digital signal to another node, for example, the second electrical node. The digital signal may be transmitted to another node via the third connector data contact. In one embodiment, the digital signal can be broadcast throughout multiple electrical nodes with each node sensitive to the header.

Unless otherwise indicated, all numbers expressing feature sizes, amounts, and physical properties used in the specification and claims are to be understood as being modified by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

### LIST OF ILLUSTRATIVE EMBODIMENTS

1. A first electrical node for forming daisy-chain buses with simultaneous serial data transmission and power transmission, comprising:

a housing,

a first connector adjacent the housing, the first connector operable to mate with a computing device;

a second connector adjacent the housing, the second connector comprises:
a second connector power contact,
a second connector data contact; and

a controller circuit comprising:
a digital-to-analog circuit that is electrically coupled to the first connector,
a processor,
a memory communicatively coupled to the processor, wherein the memory comprises instructions, that, when executed by the processor, cause the processor to:
   receive a digital signal from the second connector data contact;
   determine whether a portion of the digital signal is intended for the first electrical node; and
   convert, with the digital-to-analog circuit, the portion of the digital signal to an analog signal based on whether the portion is intended for the first electrical node without affecting the digital signal received.
      2. The first electrical node of embodiment 1, a third connector adjacent the housing, the third connector comprises:
a third connector power contact, wherein, the second connector power contact is electrically coupled to the third connector power contact;
a third connector data contact.
   3. The first electrical node of embodiment 2, wherein the second connector is a USB connector.
   4. The first electrical node of embodiment 2 or 3, wherein the controller circuit further comprises:

a power management circuit electrically coupled to the second connector power contact and the third connector power contact configured to draw power from the second connector power contact to power the controller circuit.

5. The first electrical node of embodiment 4, wherein the power management circuit comprises a battery.
6. The first electrical node of embodiment 4 or 5, wherein the power management circuit comprises a capacitor.
7. The first electrical node of any of embodiments 4 to 6, wherein a voltage drop between the second connector power contact and the third connector power contact is less than 1 volt.
8. The first electrical node of any of the preceding embodiments, wherein the second connector power contact or third connector power contact is a positive supply voltage, negative supply voltage, ground wire, or combinations thereof.
9. The first electrical node of any of the preceding embodiments, wherein the second connector data contact or third connector data contact comprises a read wire, a write wire, a common wire or combinations thereof.
10. The first electrical node of any of the preceding embodiments, wherein the controller circuit is configured to transmit the digital signal through the third connector data contact.
11. The first electrical node of any of the preceding embodiments, wherein the first connector comprises a first connector data contact,

wherein the controller circuit comprises an analog-to-digital circuit coupled to the first connector data contact and the second connector data contact such that a second analog signal is converted to a second digital signal and transmitted through the second connector data contact or third connector data contact.

12. The first electrical node of any of the preceding embodiments, wherein the second connector receives digital data from a receiver device and is communicatively coupled to the controller circuit.
13. The first electrical node of any of the preceding embodiments, further comprising a fourth connector, the fourth connector configured to electrically couple to the receiver device.
14. The first electrical node of embodiment 13, wherein the fourth connector is within the housing.
15. The first electrical node of any of the preceding embodiments, wherein the second connector power contact is configured to receive power from a power supply.
16. The first electrical node of embodiment 15, wherein the second connector data contact does not receive a digital signal when the second connector power contact receives power from the power supply.
17. The first electrical node of any of the preceding embodiments, further comprising a fifth connector operable to mate with a medical monitor, the controller circuit comprises:
a digital-to-analog circuit that is electrically coupled to the fifth connector,
wherein the memory comprises instructions, that, when executed by the processor, cause the processor to:
   receive a digital signal from the second connector data contact or the fourth connector;
   determine whether a portion of the digital signal is intended for at least one of the fifth connector and the first connector.
      18. The first electrical node of any of the preceding embodiments, wherein the computing device is a medical monitor.
      19. The first electrical node of embodiment 18, wherein the analog signal is selected from the group consisting of: ECG signal, SPO2 signal, temperature signal, blood pressure signal, and combinations thereof.
      20. A system, comprising:

the first electrical node of any of embodiments 1 to 19,

a receiver device having a receiver connector that couples to the first electrical node, wherein the receiver device is configured to receive physiological parameters wirelessly.

21. The system of embodiment 20, wherein the receiver connector is configured to be removable from the second connector.
22. The system of embodiment 20 or 21, wherein the receiver device comprises a receiver housing and wireless circuitry but not digital to analog conversion capability, the receiver housing is arranged to nestle inside of the housing of the first electrical node such that the receiver connector mates with the second connector.
23. The system of embodiment 22, wherein the receiver device, when nestled inside of the housing, does not cause mechanical interference with any other port in a linear configuration.
24. The system of embodiment 23, wherein the receiver device is configured to be oriented in a vertical orientation when the first electrical node is inserted into the computing device.
25. The system of any of embodiments 20 to 24, wherein the receiver device is powered through the first electrical node via the receiver connector.
26. The system of any of embodiments 20 to 25, further comprising:

a second electrical node having the components of the first electrical node, the first connector data contact of the second electrical node is configured to electrically couple to the second connector data contact of the first electrical node such that digital signals from the receiver device at a first electrical node are received by the second electrical node.

27. The system of embodiment 26, wherein the third connector power contact of the second electrical node is configured to couple to the second connector power contact of the first electrical node.
28. The system of any of embodiments 20 to 27, further comprising:

a third electrical node having the components of the first electrical node, the third connector data contact of the third electrical node is configured to electrically couple to the second connector data contact of the first electrical node such that digital signals from the receiver at a first electrical node are received by the third electrical node.

29. The system of any of embodiments 20 to 28, wherein the receiver device is communicatively coupled to a sensor device configured to monitor one or more physiological parameters of a mammalian subject.
30. The system of any of embodiments 20 to 29, further comprising a medical monitor.
31. The system of embodiment 30, wherein the medical monitor comprises a display that displays one or more physiological parameters of the mammalian subject.
32. The system of any of embodiments 20 to 31, further comprising a first cable to electrically couple the second connector of the first electrical node to a power supply, wherein digital signals are not transmitted through the first cable.
33. The system of any of embodiments 20 to 32, further comprising a second cable to electrically couple the third connector of the first electrical node with the second connector of the second electrical node.
34. The system of any of embodiments 20 to 33, further comprising a third cable to electrically couple the third connector of the second electrical node with the second connector of the third electrical node.

1. A first electrical node for forming daisy-chain buses with simultaneous serial data transmission and power transmission, comprising:
a housing,
a first connector adjacent the housing, the first connector operable to mate with a computing device;
a second connector adjacent the housing, the second connector comprises:
a second connector power contact,
a second connector data contact; and
a controller circuit comprising:
a digital-to-analog circuit that is electrically coupled to the first connector,
a processor,
a memory communicatively coupled to the processor, wherein the memory comprises instructions, that, when executed by the processor, cause the processor to:
receive a digital signal from the second connector data contact;
determine whether a portion of the digital signal is intended for the first electrical node; and
convert, with the digital-to-analog circuit, the portion of the digital signal to an analog signal based on whether the portion is intended for the first electrical node without affecting the digital signal received.

2. The first electrical node of claim 1, a third connector adjacent the housing, the third connector comprises:
a third connector power contact, wherein, the second connector power contact is electrically coupled to the third connector power contact;
a third connector data contact.

3. The first electrical node of claim 2, wherein the controller circuit further comprises:
a power management circuit electrically coupled to the second connector power contact and the third connector power contact configured to draw power from the second connector power contact to power the controller circuit.

4. The first electrical node of claim 3, wherein a voltage drop between the second connector power contact and the third connector power contact is less than 1 volt.

5. The first electrical node of claim 2, wherein the second connector power contact or third connector power contact is a positive supply voltage, negative supply voltage, ground wire, or combinations thereof.

6. The first electrical node of claim 2, wherein the second connector data contact or third connector data contact comprises a read wire, a write wire, a common wire or combinations thereof.

7. The first electrical node of claim 2, wherein the controller circuit is configured to transmit the digital signal through the third connector data contact.

8. The first electrical node of claim 2, wherein the first connector comprises a first connector data contact,
wherein the controller circuit comprises an analog-to-digital circuit coupled to the first connector data contact and the second connector data contact such that a second analog signal is converted to a second digital signal and transmitted through the second connector data contact or third connector data contact.

9. The first electrical node of claim 2, further comprising a fourth connector, the fourth connector configured to electrically couple to a receiver device.

10. The first electrical node of claim 9, wherein the fourth connector is within the housing.

11. The first electrical node of claim 1, wherein the second connector power contact is configured to receive power from a power supply, wherein the second connector data contact does not receive a digital signal when the second connector power contact receives power from the power supply.

12. The first electrical node of claim 9, further comprising a fifth connector operable to mate with a medical monitor, the controller circuit comprises:
a digital-to-analog circuit that is electrically coupled to the fifth connector,
wherein the memory comprises instructions, that, when executed by the processor, cause the processor to:
receive a digital signal from the second connector data contact or the fourth connector;
determine whether a portion of the digital signal is intended for at least one of the fifth connector and the first connector.

13. The first electrical node of claim 1, wherein the computing device is the medical monitor, wherein the analog signal is selected from the group consisting of: ECG signal, SPO2 signal, temperature signal, blood pressure signal, and combinations thereof.

14. A system, comprising:
a first electrical node, comprising:
a housing,
a first connector adjacent the housing, the first connector operable to mate with a computing device;
a second connector adjacent the housing, the second connector comprises:
a second connector power contact,
a second connector data contact; and
a controller circuit comprising:
a digital-to-analog circuit that is electrically coupled to the first connector,
a processor,
a memory communicatively coupled to the processor, wherein the memory comprises instructions, that, when executed by the processor, cause the processor to:
receive a digital signal from the second connector data contact;
determine whether a portion of the digital signal is intended for the first electrical node; and
convert, with the digital-to-analog circuit, the portion of the digital signal to an analog signal based on whether the portion is intended for the first electrical node without affecting the digital signal received; and
a receiver device having a receiver connector that couples to the first electrical node, wherein the receiver device is configured to receive physiological parameters wirelessly.

15. The system of claim 14, wherein the receiver device comprises a receiver housing and wireless circuitry but not digital to analog conversion capability, the receiver housing is arranged to nestle inside of a housing of the first electrical node such that the receiver connector mates with the second connector.

16. The system of claim 14, wherein the receiver device is powered through the first electrical node via the receiver connector.

17. The system of claim 14, further comprising:
a second electrical node having components of the first electrical node, the first connector data contact of the second electrical node is configured to electrically couple to the second connector data contact of the first electrical node such that digital signals from the receiver device at a first electrical node are received by the second electrical node.

18. The system of claim 17, wherein the third connector power contact of the second electrical node is configured to couple to the second connector power contact of the first electrical node.

19. The system of claim 17, further comprising:
a third electrical node having the components of the first electrical node, the third connector data contact of the third electrical node is configured to electrically couple to the second connector data contact of the first electrical node such that digital signals from the receiver at the first electrical node are received by the third electrical node.

20. A method, comprising:
receiving a digital signal from a connector data contact from a first electrical node;
determining whether a portion of the digital signal is intended for the first electrical node;
converting, with the digital-to-analog circuit, the portion of the digital signal to an analog signal based on whether the portion is intended for the first electrical node without affecting the digital signal received; and
transmitting the analog signal to a computing device.
